(19) United States
(12) Patent Application Publication
(10) Pub. No.: US 2021/0100613 A1
(43) Pub. Date: Apr. 8, 2021

(54) RETURN ELECTRODE COMPRESSION SLEEVE

(71) Applicant: COVIDIEN LP, Mansfield, MA (US)

(72) Inventors: JACOB C. BARIL, NORWALK, CT (US); SAUMYA BANERJEE, HAMDEN, CT (US)

(21) Appl. No.: 16/594,967

(22) Filed: Oct. 7, 2019

(51) Int. Cl. A61B 18/16; A61B 18/12

(52) U.S. Cl. CPC A61B 18/16; A61B 2017/00867; A61B 18/1206

(57) ABSTRACT

A return electrode includes a removable sleeve having an outer peripheral surface and an inner peripheral surface configured to slide over a patient's limb. The removable sleeve also includes at least one electrically conductive pad that is operably associated with the inner peripheral surface, and adapted to connect to an electrosurgical generator. At least one sensor is associated with the sleeve and configured to measure a current level for each electrically conductive pad, such that the current levels of each electrically conductive pad is input into a computer algorithm configured to control the power output of the electrosurgical generator. A compression mechanism is disposed within the sleeve to compress the outer peripheral surface against the patient's limb.

### FIELD

The present disclosure is directed to an electrosurgical apparatus and method and, more particularly, is directed to a patient return electrode sleeve and a method for performing monopolar surgery using the same.

### BACKGROUND

During electrosurgery, a source or active electrode delivers energy, such as radio frequency energy, from an electrosurgical generator to cut and coagulate tissue, while a return electrode is used to safely redirect current from the active electrode back to the electrosurgical generator across the patient's body.

Historically, return electrodes were in the form of large metal plates placed on the body that were covered with a conductive jelly. More recently, adhesive electrodes were developed that include a metal foil covered with a conductive jelly or a conductive adhesive. In either of these instances, the return electrodes tended to be messy, were prone to patient slippage and, in some instances, areas of non-compliance or non-contact areas were formed which reduced the overall effectiveness of the return electrode. For example, when slippage occurs the contact area between the electrode and the patient is decreased, thereby increasing the current density within the portion of the electrode that remains in contact and, in turn, increases the heat applied to the tissue. This may increase the heat associated with the patient area under the adhered portion of the return electrode. Additionally, the contact area between the electrode and the patient can be affected by external forces, for example if the electrode were to be unintentionally shifted by a nurse or surgeon during treatment.

Return Electrode Monitors (REMs), were developed to sense the change in impedance so that when the percentage increase in impedance exceeds a predetermined value or the measured impedance exceeds a threshold level, the electrosurgical generator is shut down to reduce the chances of harming the patient. Typically, REM Monitors were associated with one or more return electrodes pads placed under the patient. However, as mentioned above, return electrode pads do have drawbacks.

### SUMMARY

Provided in accordance with aspects of the present disclosure is a return electrode, configured for use within a removable sleeve. The removable sleeve includes an outer peripheral surface and an inner peripheral surface, the inner peripheral surface configured to slide over a patient's limb.

In an aspect of the present disclosure, one or more electrically conductive pads operably associated with the inner peripheral surface, the electrically conductive pad(s) adapted to connect to an electrosurgical generator.

In another aspect of the present disclosure, one or more sensors are configured to measure a current level of each electrically conductive pad, the current levels of each electrically conductive pad being input into a computer algorithm configured to control the power of the electrosurgical generator based upon the output of the computer algorithm.

In still another aspect of the present disclosure, a compression mechanism compresses the outer peripheral surface of the removable sleeve against the patient's limb.

In yet another aspect of the present disclosure, two or more electrically conductive pads are operably associated with the inner peripheral surface of the removable sleeve.

In still yet another aspect of the present disclosure, the outer peripheral surface is integrally associated with the compression mechanism.

In another aspect of the present disclosure, in the outer peripheral surface includes a compression material including spandex, nylon-spandex, elastane, polyether-polyurea copolymer, microfiber and/or silk.

In an aspect of the present disclosure, the compression mechanism includes an inflatable material operably associated with the outer peripheral surface of the removable sleeve.

In still another aspect of the present disclosure, the compression mechanism includes a selectively deformable material operably associated with the outer peripheral surface, the selectively deformable material configured to deform when introduced to temperature, energy, and/or light.

In an aspect of the present disclosure, the selectively deformable material includes a shape memory metal, a shape memory polymer, an electro-memory materials, and/or a light memory materials.

In another aspect of the present disclosure, a power cord is operably associated with the removable sleeve, the power cord configured to operably connect the the conductive pad(s) with the electrosurgical generator.

In still another aspect of the present disclosure, the sensor cooperates with a variable impedance controller that regulates an impedance level based upon the output from the computer algorithm.

In yet another aspect of the present disclosure, the variable impedance controller, the sensor, and/or the computer algorithm are housed within the electrosurgical generator.

In still yet another aspect of the present disclosure, the electrosurgical generator is coupled to the variable impedance controller, the sensor, and/or the computer algorithm and operable to adjust the amount of current provided based upon a control signal from the variable impedance controller.

In another aspect of the present disclosure, each conductive pad includes a plurality of variable impedances.

In an aspect of the present disclosure, the variable impedance controller is selectively adjustable to a predetermined level prior to delivery of current.

In another aspect of the present disclosure, the variable impedance is a rheostat or a potentiometer.

In still another aspect of the present disclosure, the variable impedance controller utilizes proportional-integral-derivative (PID) control.

In yet another aspect of the present disclosure, the variable impedance controller utilizes digital control.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is a schematic illustration of a monopolar electrosurgical system with a return electrode;

FIG. 1B is a detail of a leading edge of a return electrode;

FIG. 2A is a perspective view of an embodiment of a removable sleeve in use on a patient's left forearm;

FIG. 2B is a perspective view of an embodiment of a removable sleeve in use on a patient's right leg;

FIG. 3A is a perspective view of the removable sleeve of FIG. 2A;

FIG. 3B is an exploded view of the removable sleeve of FIG. 3A;

FIG. 3C is a cross-sectional view of the removable sleeve, of FIG. 3A across line C-C;

FIG. 4A is a flow chart outlining the steps in the process of current redistribution across the inner peripheral surface of a removable sleeve; and

FIG. 4B is a flow chart outlining the steps in an alternate process of current regulation.

### DETAILED DESCRIPTION

Embodiments of the presently-disclosed electrosurgical return electrode and method of using the same are described below with reference to the accompanying drawing figures wherein like reference numerals identify similar or identical elements. In the following description, well-known functions or constructions are not described in detail to avoid obscuring the disclosure in unnecessary detail. In addition, terms such as “above”, “below”, “forward”, “rearward”, etc. refer to the orientation of the figures or the direction of components and are simply used for convenience of description.

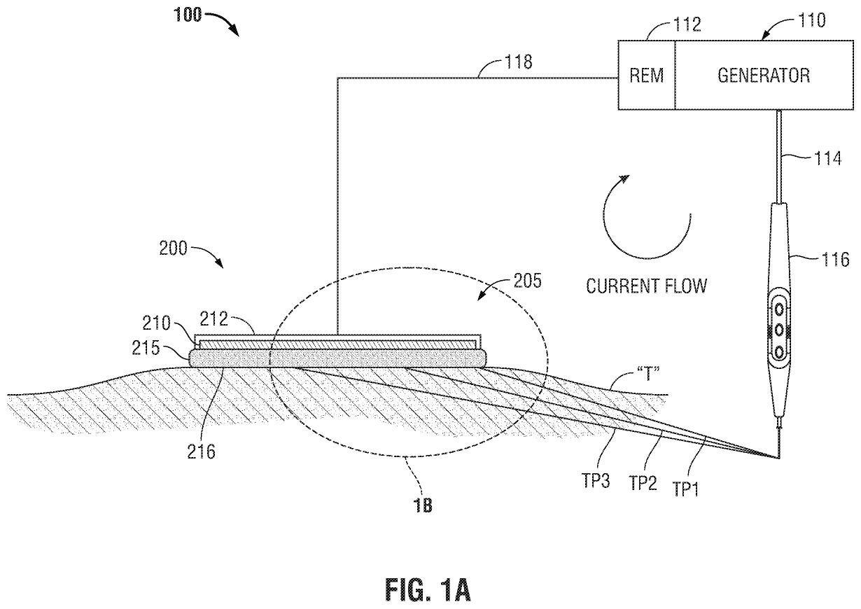

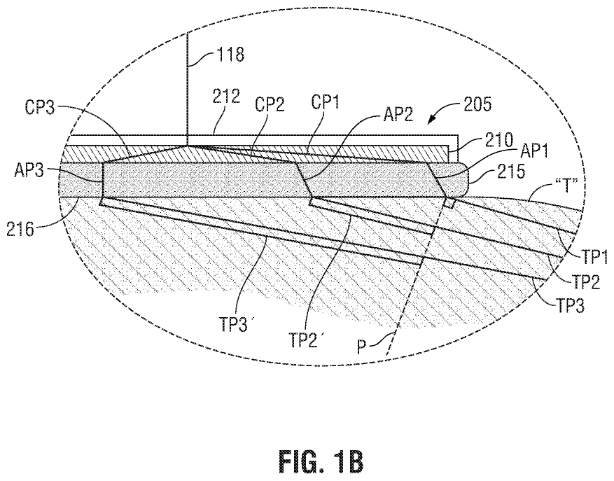

Referring initially to FIG. 1A, a schematic illustration of a monopolar electrosurgical system 100 is shown. The electrosurgical system 100 generally includes a typical return electrode pad 200, an electrosurgical generator 110, a surgical instrument 116 (e.g., an active electrode) and a return electrode monitor (REM) 112. In FIG. 1A and in the figures hereinbelow, return electrode pad 200 is illustrated in contact with patient tissue “T”. Generally, electrosurgical energy is supplied to the active electrode 116 by the generator 110 through a supply cable 114 to treat tissue (e.g., cut, coagulate, blend, etc.). The return electrode pad 200 acts as a return path for energy delivered by the active electrode 116 to patient tissue “T”. Energy returns back to the electrosurgical generator 110 via a return cable 118.

While FIGS. 1A and 1B depict cross-sections of return electrode pad 200 it is within the scope of the disclosure for the return electrodes to have any suitable regular or irregular shape.

In the embodiments illustrated in FIGS. 1A-B, return electrode pad 200 is formed of a conductive layer 210 engaged on the top with an insulating layer 212 and on the bottom with a contact layer 215. Conductive layer 210 connects to generator 110 by return cable 118 in any suitable manner

Contact layer 215 is formed of a gel or adhesive configured to couple to patient tissue “T” and can be made from, but is not limited to, a polyhesive adhesive, conductive hydrogel, a Z-axis adhesive or a water-insoluble, hydrophilic, pressure- sensitive adhesive. The portion of the contact layer 215 in contact with a patient tissue “T” is a patient-contacting surface 216 that is configured to ensure an optimal contact area between the return electrode pad 200 and the patient tissue “T”. In addition, contact layer 215 provides ionic conductive contact with the skin to transfer energy out of the body.

A leading edge 205 of the return electrode 200 is that portion of the return electrode pad 200 positioned closest to the active electrode 116. Leading edge 205 is defined in this disclosure not as a single point but as a general portion of the return electrode pad 200 positioned closest to the active electrode 116.

In use, the current applied by the active electrode 116 travels through various tissue paths between the active electrode 116 and the return electrode pad 200. The amount of current supplied by the active electrode 116 is typically equal to the amount of current received by the return electrode pad 200. The only difference between the active electrode 116 and the return electrode pad 200 is the amount of area in which the current is conducted. Concentration of electrons at the active electrode 116 is high due to the small surface area of the active electrode 116, which results in high current density and generation of heat, while the large surface area of the return electrode pad 200 disperses the same current over the large contacting surface 216 resulting in a low current density and little production of heat.

Electric charge passing between the active electrode 116 and the return electrode pad 200 will travel along various paths in patient tissue “T” and will seek the path with the lowest impedance. With reference to FIGS. 1A and 1B, three tissue paths (TP1), (TP2) and (TP3) are provided for illustrating tissue paths with varying impedances. However, any number of suitable paths may be utilized for conducting current through tissue “T”.

Tissue path one (TP1) is a path in patient tissue “T” between the active electrode 116 and the leading edge 205 of return electrode pad 200. Tissue path two (TP2) and tissue path three (TP3) are paths in patient tissue “T” between the active electrode 116 and a portion of the return electrode pad 200 away from the leading edge 205 of the return electrode pad 200.

The total impedance of a given pathway between the active electrode 116 and the return cable 118, through the return electrode 200, is determined by combining the impedance of the tissue pathway and the impedance of the various layers of the return electrode pad 200. As illustrated in FIG. 1B, the impedance of the first path equals the sum of the impedance of the first tissue path (TP1), the impedance of the first adhesive path (AP1) through the contact layer 215 and the impedance of the first conductive path (CPI) through the conductive layer 220. Similarly, the impedance of the second path equals the sum of the impedance of the second tissue path (TP2), the impedance of the second adhesive path (AP2) and the impedance of the second conductive path (CP2). Finally, impedance of the third path equals the sum of the impedance of the third tissue path (TP3), the impedance of the third adhesive path (AP3) and the impedance of the third conductive path (CP3).

In comparing the impedance of the various portions of the three illustrative current pathways, the impedance of adhesive paths (AP1), (AP2) and (AP3) and the impedance of conductive paths (CP1), (CP2) and (CP3) are substantially the same regardless of the tissue path selected. In addition, the impedance of adhesive path (AP1), (AP2) and AP3 and the impedance of a conductive path (CP1), (CP2) and (CP3) are generally small in comparison to the impedance of a tissue path (TP1), (TP3) and (TP3) and are therefore negligible with respect to the impedance of each respective tissue path (TP1), (TP2) and (TP3). Therefore, the current density at any point on the contacting surface 216 is generally dependent on the impedance of the tissue path.

As illustrated by perpendicular “P” drawn from first tissue path (TP1) in FIG. 1B, the lengths of the second and third tissue paths (TP2) and (TP3) are longer than first tissue path (TP1) by lengths of (TP2′) and (TP3′), respectively. This additional length (TP2′) and (TP3′) in tissue adds additional impedance to second and third tissue paths (TP2) and (TP3), thus resulting in a higher current density at the leading edge 205 and a reduction in current density away from leading edge 205.

This phenomenon, known as “Leading Edge Effect,” results in the concentration of energy and heat at the leading edge 205 of the return electrode pad 200 and heating imbalance across the return electrode pad 200. Leading Edge Effect may result in serious injury to skin under the leading edge 205 if patient tissue “T” is heated beyond the point where circulation of blood can cool the tissue.

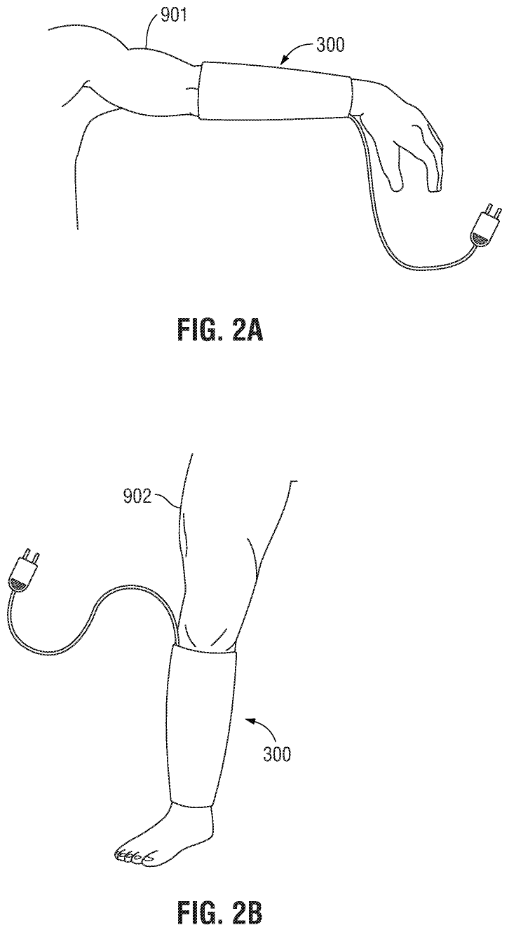

With reference to FIGS. 2A-B, a return electrode is shown in the form of a removable sleeve 300 that can be worn by a patient during treatment to ensure proper contact is maintained between the patient and the removable sleeve 300 at all times, and to increase the amount of surface area in contact between the removable sleeve 300 and the patient. More specifically, the removable sleeve 300 is configured to be worn on any of the patient's limbs. Depending on where on the patient's body a surgeon needs to access during treatment, the removable sleeve 300 can be positioned on whichever limb the surgeon considers most convenient. For example, in FIG. 2A the surgeon may need to access a location on the left portion of the upper torso, therefore the removable sleeve 300 may be worn on the patient's left arm 901. Likewise in FIG. 2B the surgeon may need to access a location on the right side of the lower abdomen, therefore the removable sleeve 300 may be worn on the patient's right leg 902.

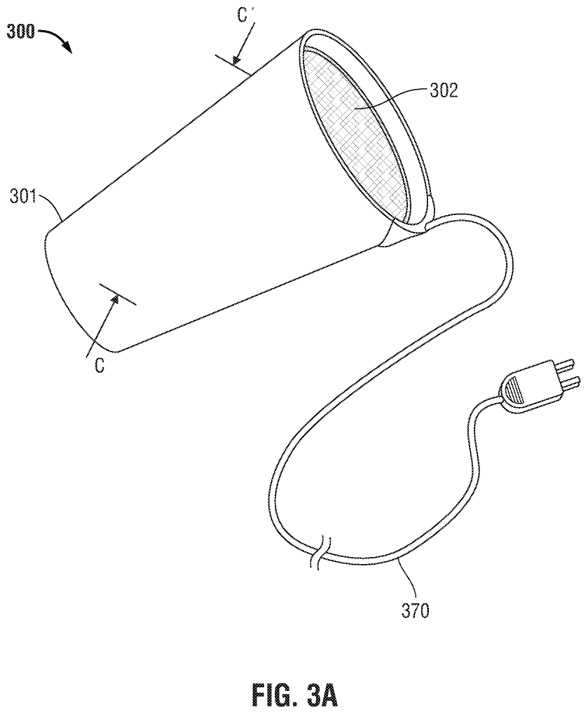

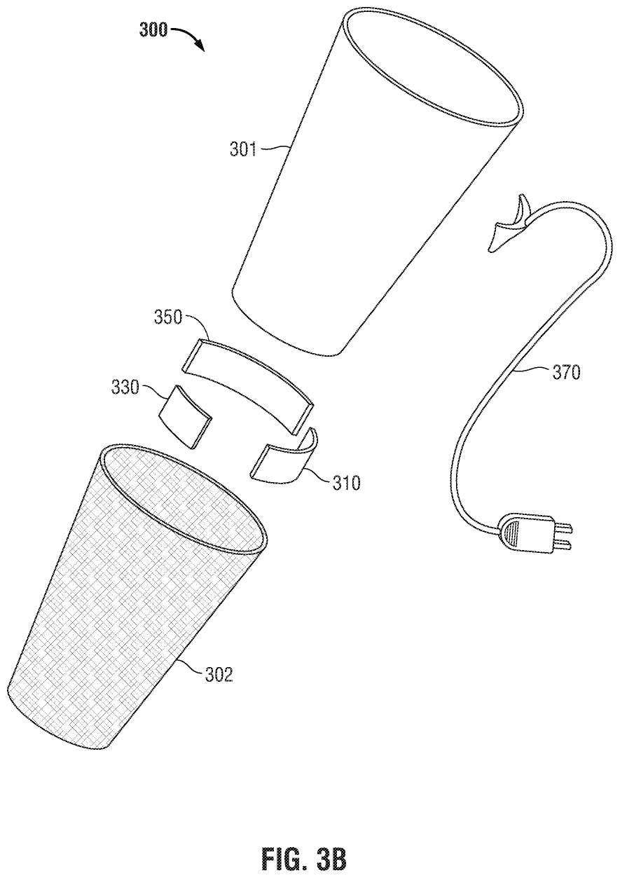

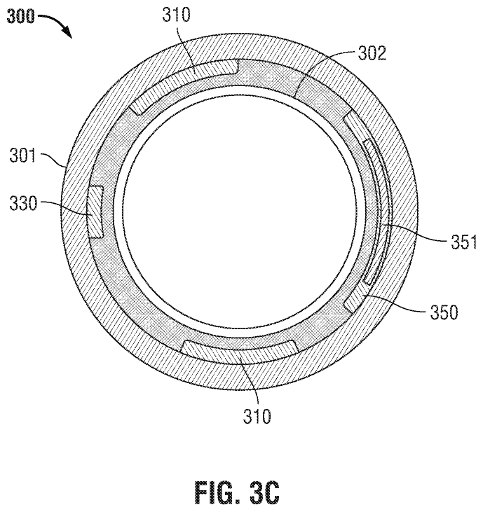

Referring now to FIGS. 3A-C, the removable sleeve 300 includes an outer peripheral surface 301 and an inner peripheral surface 302, where the inner peripheral surface 302 is configured to slide over a patient's limb. In use, the removable sleeve 300 is pulled over the patient's limb such that the inner peripheral surface 302 is in direct contact with the patient's skin. One or more electrically conductive pads 310 is adapted to connect to the electrosurgical generator 110 (FIG. 1A) via a dual purpose return cable/power cord 370 operably associated with the removable sleeve 300. Each electrically conductive pad 310 is operably associated with the inner peripheral surface 302 to allow for the contact area between the electrically conductive pad 310 and the patient to be extended from the surface area of the electrically conductive pad 310 to encompass the entire surface area of inner peripheral surface 302. In some embodiments, each removable sleeve 300 includes two or more electrically conductive pads 310 to more efficiently redistribute the returning current across inner peripheral surface 302. Each conductive pad 310 may further include a plurality of variable impedances 362.

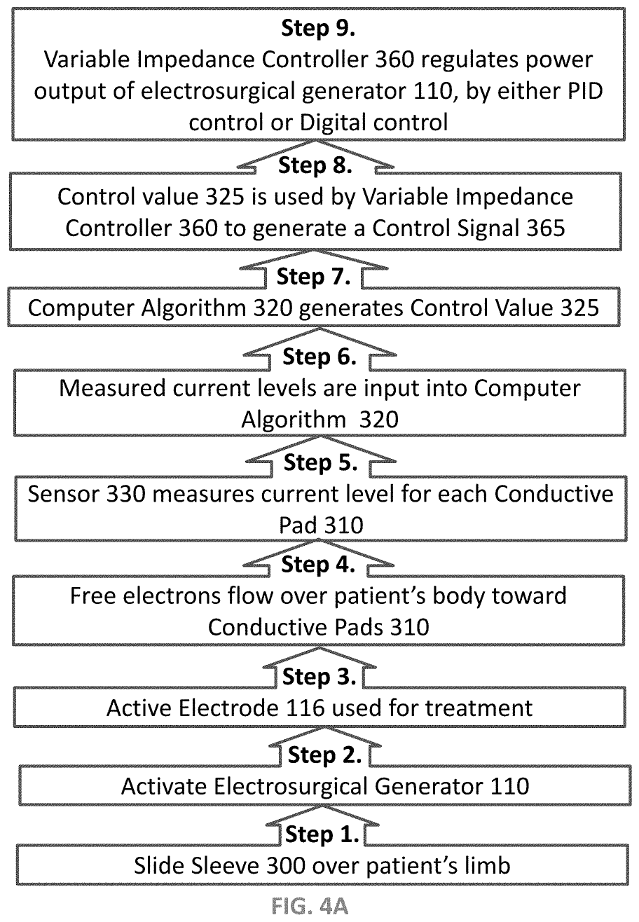

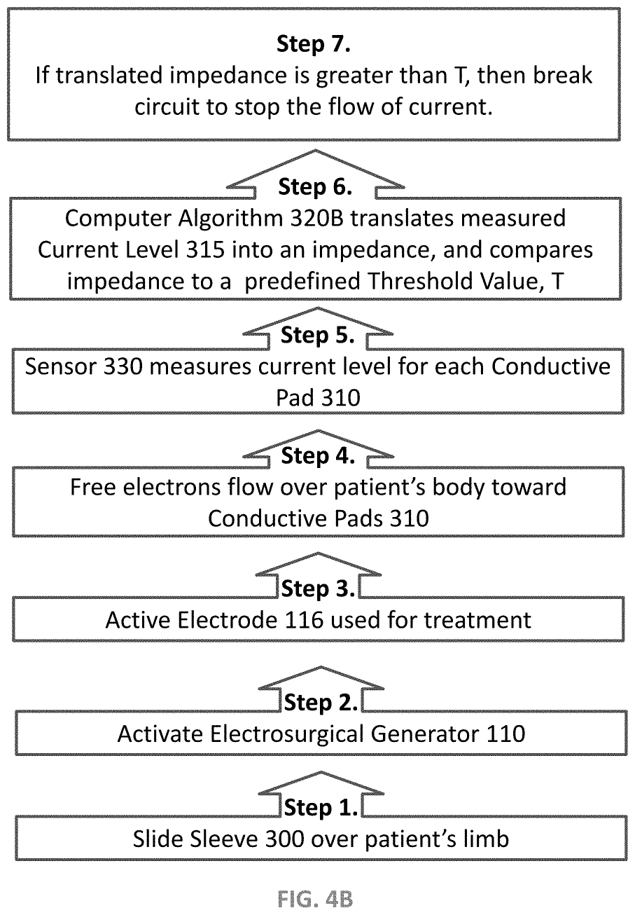

Referring now to the flow chart in FIG. 4A the process is shown by which current is evenly redistributed across one or more electrically conductive pads 310 by a variable impedance controller 360. After placing the removable sleeve 300 onto the patient in step 1, activating the electrosurgical generator 110 in step 2 and beginning treatment with an active electrode 116 in step 3, current will flow along the surface of the patient's body toward the conductive pads 310 and through the return cable 370 en route back to the electrosurgical generator 110 as shown in step 4. While flowing through the electrically conductive pads 310 the current encounters one or more sensors 330 disposed within the removable sleeve 300 to measure a current level 315 of each electrically conductive pad 310. The sensor(s) 330 is operably associated with the inner peripheral surface 302 as shown in steps 5 and 6.

Once measured, the current levels 315 for each electrically conductive pad 310 is fed into a computer algorithm 320 which, in turn, generates a control value 325, that can be used by the variable impedance controller 360 to generate a control signal 365 as shown in steps 7 and 8. More specifically, the variable impedance controller 360 is configured to transmit a control signal 365 based upon the output of the computer algorithm 320 to regulate the amount of power provided by the electrosurgical generator to the active electrode 116 in step 2.

In steps 8 and 9, the variable impedance controller 360 utilizes the control signal 365 by either proportional-integral-derivative (PID) control, or digital control to regulate the amount of power provided by the electrosurgical generator 110 to the active electrode, which, in turn, allows for an even redistribution of current flow to each of the electrically conductive pads 310. Additionally, the current is redistributed such that no one electrically conductive pad 310 is overloaded thereby reducing instances of electrically conductive pads 310 overheating and burning the patient.

FIG. 4B shows another flow chart illustrating an alternative process. The two processes are the same for steps 1-5. In step 6 rather than redistributing current flow across multiple electrically conductive pads 310, once the sensor 330 measures the current level 315 for each electrically conductive pad 310 in step 5, a second computer algorithm 320B determines whether (or not) the measured current level 315 corresponds to a level of impedance beyond a predetermined threshold value, T, for the electrically conductive pad 310. If the level of impedance exceeds the threshold value T, then the second computer algorithm 320B is configured to immediately disconnect the circuit to stop the flow of current to thereby prevent overheating of the electrically conductive pad 310. In practice, the threshold value used by the second computer algorithm 320B will need to be higher or lower depending on the amount of energy required by the active electrode and the number of electrically conductive pads 310 available. Therefore, this predetermined threshold value T, is selectively adjustable prior to delivery of current 315, and the role of the variable impedance controller 360 can be satisfied by either a rheostat or a potentiometer.

In embodiments, the electrosurgical generator 110 is an external device connected to the return electrode by a return cable 370. Any of the variable impedance controller 360, sensor 330, and computer algorithm 325 can also be housed within and operably coupled to the electrosurgical generator 110 to adjust the amount of current 315 provided based upon a control signal 365 from the variable impedance controller 360.

Referring back to FIGS. 3B and 3C, the respective exploded and cross-sectional views of the removable sleeve 300 show where a compression mechanism 350 may be disposed within the removable sleeve. The compression mechanism 350 may be configured to expand within the removable sleeve 300 such that the outer peripheral surface 301 of the removable sleeve 300 is compressed against the patient's limb.

To facilitate compression of the removable sleeve 300 against the patient's limb, the compression mechanism 350 may include an inflatable material configured to expand the inner volume of the removable sleeve 300, while the outer peripheral surface 301 may be comprised of a compression material configured to restrict expansion of the removable sleeve 300. This combination of expansion from the inflatable material and restriction from the compression material creates an inward compressive force on the limb of the patient wearing the removable sleeve 300. In embodiments, the compression mechanism 350 is an air pump disposed within the removable sleeve 300 with a control interface disposed on the outer peripheral surface 301, such that the air pump can be manually or automatically inflated. The compression material may include spandex, nylon-spandex, elastane, polyether-polyurea copolymer, or the like and the outer peripheral surface 301 may include silk or a microfiber material. In embodiments, the compression mechanism 350 may be a simple belt coupled to a fastener disposed within the removable sleeve 300, such that pulling the belt through the fastener contracts the belt around the patient's arm to hold the sleeve 300 in place.

The outer peripheral surface 301 may be configured to introduce any one of temperature, energy, or light to a selectively deformable material 351 disposed within the compression mechanism 350. When exposed to any one of temperature, energy, or light, the selectively deformable material 351 reacts to the stimulation by reshaping itself into a new expanded form, which can add to the compressive force acting on the limb of the patient wearing the removable sleeve 300. In this way the outer peripheral surface 301 is operably associated with the selectively deformable material 351 and integrally associated with the compression mechanism 350. In embodiments, the selectively deformable material 351 includes a shape memory metal, shape memory polymer, electro-memory materials, and/or light memory materials.

What is claimed is:

1. A return electrode, comprising:
a removable sleeve including an outer peripheral surface and an inner peripheral surface, the inner peripheral surface configured to slide over a patient's limb, the removable sleeve including:
at least one electrically conductive pad operably associated with the inner peripheral surface, the at least one electrically conductive pad adapted to connect to an electrosurgical generator;
at least one sensor configured to measure a current level of each at least one electrically conductive pad, the current levels of each at least one electrically conductive pad being input into a computer algorithm configured to control the power of the electrosurgical generator based upon the output of the computer algorithm; and
a compression mechanism for compressing the outer peripheral surface of the removable sleeve against the patient's limb.

2. The return electrode sleeve according to claim 1, wherein at least two electrically conductive pads are operably associated with the inner peripheral surface of the removable sleeve.

3. The return electrode sleeve according to claim 1, wherein the outer peripheral surface is integrally associated with the compression mechanism.

4. The return electrode sleeve according to claim 3, wherein the outer peripheral surface includes a compression material including at least one of spandex, nylon-spandex, elastane, polyether-polyurea copolymer, microfiber or silk.

5. The return electrode sleeve according to claim 1, wherein the compression mechanism includes an inflatable material operably associated with the outer peripheral surface of the removable sleeve.

6. The return electrode sleeve according to claim 1, wherein the compression mechanism includes a selectively deformable material operably associated with the outer peripheral surface, the selectively deformable material configured to deform when introduced to at least one of temperature, energy, or light.

7. The return electrode sleeve according to claim 6, wherein the selectively deformable material includes at least one of a shape memory metal, shape memory polymer, electro-memory materials, or light memory materials.

8. The return electrode sleeve according to claim 1, further comprising a power cord operably associated with the removable sleeve, the power cord configured to operably connect the at least one conductive pad with the electrosurgical generator.

9. The return electrode sleeve according to claim 1, wherein the at least one sensor cooperates with a variable impedance controller that regulates an impedance level based upon the output from the computer algorithm.

10. The return electrode sleeve according to claim 9, wherein at least one of the variable impedance controller, sensor, and computer algorithm are housed within the electrosurgical generator.

11. The return electrode sleeve according to claim 9, wherein the electrosurgical generator is coupled to at least one of the variable impedance controller, sensor, and computer algorithm and operable to adjust the amount of current provided based upon a control signal from the variable impedance controller.

12. The return electrode sleeve according to claim 1, wherein each at least one conductive pad includes a plurality of variable impedances.

13. The return electrode sleeve according to claim 9, wherein the variable impedance controller is selectively adjustable to a predetermined level prior to delivery of current.

14. The return electrode sleeve according to claim 9, wherein the variable impedance is at least one of a rheostat or a potentiometer.

15. The return electrode sleeve according to claim 9, wherein the variable impedance controller utilizes proportional-integral-derivative (PID) control.

16. The return electrode sleeve according to claim 9, wherein the variable impedance controller utilizes digital control.

17. A method of performing monopolar surgery, comprising:
covering a patient's limb with a removable sleeve including an outer peripheral surface and an inner peripheral surface, the inner peripheral surface configured to slide over the patient's limb;
compressing the outer peripheral surface of the removable sleeve against the patient's limb;
measuring a current level of at least one electrically conductive pad operably associated with the inner peripheral surface of the removable sleeve; and
inputting the current level of each at least one electrically conductive pad into a computer algorithm configured to control the power of the electrosurgical generator based upon the output of the computer algorithm.

18. The method of performing monopolar surgery according to claim 17 further comprising adjusting a variable impedance level of the at least one electrically conductive pads based upon the output generated by the computer algorithm.

19. The method of performing monopolar surgery according to claim 18 further comprising: measuring the current returning to each at least one electrically conductive pad;
detecting imbalances in current by monitoring the current returning to each at least one electrically conductive pad; and
controlling the current entering each at least one electrically conductive pad using the computer algorithm and a variable impedance controller to vary impedances.

20. The method of performing monopolar surgery according to claim 19 further comprising:
setting the variable impedance controller to predetermined levels prior to delivery of current, thereby allowing for more or less current to be directed towards certain at least one electrically conductive pads.
